(19) United States
(12) Patent Application Publication
(10) Pub. No.: US 2003/0228581 A1
(43) Pub. Date: Dec. 11, 2003

(54) Diagnosis and treatment for late onset neurodegenerative disorder

(75) Inventors: Jeffrey A. Klein, Mount Desert, ME (US); Susan L. Ackerman, Bar Harbor, ME (US)

Correspondence Address: Kevin M. Farrell, One New Hampshire Avenue, Suite 350, Portsmouth, NH, 03801, US

(73) Assignee: The Jackson Laboratory, Bar Harbor, ME (US)

(21) Appl. No.: 10/164,295

(22) Filed: Jun. 6, 2002

(51) Int. Cl. C12Q 1/68

(52) U.S. Cl. 435/6

(57) ABSTRACT

Disclosed is a method for diagnosing the molecular basis for a late-onset neurodegenerative disorder in a mammal. In this method, a sample from the mammal is provided, the sample containing a nucleic acid sequence encoding the AIF. The sequence of nucleic acids in the nucleic acid sequence is determined by conventional techniques. This determined sequence of nucleic acids is then compared to a nucleic acid sequence encoding the wild-type AIF protein. Any difference between the sequence of nucleic acids determined from the two samples represents a candidate mutation. The candidate mutation is then further analyzed for an association with a decrease in expression of functional AIF protein. Such an association confirms a molecular basis for the late-onset neurodegenerative disorder in the mammal. Related methods and compositions are also disclosed.

### BACKGROUND OF THE INVENTION

Mitochondrial dysfunction can lead to generation of reactive oxygen species (ROS) and result in programmed cell death. Such alterations in mitochondrial function have been implicated in numerous neurodegenerative disorders, including amyotrophic lateral sclerosis, Parkinson's disease, Alzheimer's disease and retinal degeneration. Furthermore, the later onset for the sporadic forms of these disorders is consistent with age-related oxidative stress. In addition, cell cycle abnormalities have been reported in neurons in brain regions undergoing degeneration. Although oxidative stress plays an important role in the pathogenesis of several common human neurodegenerative disorders, there has been no direct evidence of inheritance of mutations in genes encoding either mitochondrial- or nuclear-encoded mitochondrial proteins implicated in neurodegenerative disease. The molecular events that result from oxidative stress leading to neuronal death have remained largely unknown.

### SUMMARY OF THE INVENTION

In one aspect, the present invention relates to a method for diagnosing the molecular basis for a late-onset neurodegenerative disorder in a mammal. In this method, a sample from the mammal is provided, the sample containing a nucleic acid sequence encoding the Aif. The sequence of nucleic acids in the nucleic acid sequence is determined by conventional techniques. This determined sequence of nucleic acids is then compared to a nucleic acid sequence encoding the wild-type AIF protein. Any difference between the sequence of nucleic acids determined from the two samples represents a candidate mutation. The candidate mutation is then further analyzed for an association with a decrease in expression of functional AIF protein. Such an association confirms a molecular basis for the late-onset neurodegenerative disorder in the mammal. Related methods and compositions are also disclosed.

### DETAILED DESCRIPTION OF THE INVENTION

As discussed above, late-onset neurodegenerative disorder characterized by the accumulation of ROS-induced damage, is well documented in humans, as well as other mammals. In addition, cell cycle abnormalities have been reported in neurons in brain regions undergoing degeneration. The present invention is based on the discovery of a molecular basis for this class of disorders. More specifically, as detailed in the Exemplification section which follows, the Aif gene product has been implicated in the disorder. In particular, it has been demonstrated that decreased levels of AIF expression correlate with oxidative stress caused by progressive increases in ROS-induced damage in neurons. In turn, the resultant stress leads to the unscheduled cell cycle reentry of these cells. However, neurons that re-enter the cell cycle cannot proliferate and thus, undergo apoptosis. The processes of oxidative stress and aberrant cell cycle re-entry are critically intertwined in terminally differentiated neurons, and their correlation with decreased AIF expression levels provides a novel molecular mechanism by which free radical damage leads to neuronal death and, ultimately, to development of late-onset neurodegenerative disorder.

These findings are directly applicable to methods for determining the molecular basis for disease development. One aspect of the present invention relates to a method for diagnosing the molecular basis for late-onset neurodegenerative disorder in a mammal by examination of the mammal for the presence of Aif gene mutations or polymorphisms. The term late-onset neurodegenerative disorder as used herein includes, without limitation, age-related ROS-induced oxidative stress, neuronal cell loss, retinal degeneration, aberrant cell cycle re-entry of terminally differentiated neurons, lipid peroxidation, and DNA oxidative damage.

The method involves isolation of nucleic acid from the mammal to be diagnosed, followed by the analysis of the nucleic acid for differences in Aif sequence compared to the corresponding wild-type Aif gene. Once the genotypic difference of the mammal to be diagnosed is determined, the difference will represent a candidate mutation or polymorphism that will be further analyzed for its association with decreased expression of functional AIF protein. Such association will confirm a molecular basis for late-onset neurodegenerative disorder in the mammal.

The sample provided from the mammal may be any sample containing nucleic acid sequence encoding AIF protein, such as a sample of cells. If the mammal to be diagnosed is human, the nucleic acid sequence encoding Aif is available in the Genbank database. For other mammals of interest, where Aif sequence information is not available, such information must be determined using techniques of cloning and sequencing known in the art. For example, expression library from any mammal can be screened to obtain Aif sequence specific to that mammal using labeled human Aif sequence as probe (mouse Aif ortholog sequence available in Genbank accession #BC003292). Also, for example, oligonucleotide primers designed based on human or mouse Aif sequence can be used to amplify Aif cDNA or Aif genomic DNA from other mammals using techniques based on PCR and known in the art.

The candidate mutation can be further analyzed for an association with a decrease in expression of functional AIF protein in a variety of ways which would be recognized by those skilled in the art. For example, Northern blot analysis can be used to directly assay mRNA transcript levels. Alternatively, levels of the AIF protein itself can be monitored directly. This can be accomplished, for example, using a monoclonal antibody specific for AIF in an affinity-based assay. The determination of such an association serves to confirm a molecular basis for the late-onset neurodegenerative disorder in the mammal.

In another aspect, the present invention relates to a method for diagnosing the molecular basis for a late-onset neurodegenerative disorder in a mammal to be diagnosed. A biopsy sample containing cells which would manifest the AIF dysfunction, if present, is initially provided. Such a sample may be obtained post-mortem for purposes of cataloguing specific mutations responsible for the disorder. As demonstrated in the Exemplification section which follows, brain, retinal and liver cells were shown to exhibit such AIF dysfunction in a mouse model system. Expression level of AIF protein in these cells is determined by conventional techniques, such as those discussed above. The AIF levels determined are then compared to an otherwise identical determination from the cells of an individual known to be unaffected by a late-onset neurodegenerative disorder, a substantial decrease in the level of expression of the AIF protein in the cells of the mammal to be diagnosed, as compared to the level from the cells of the mammal known to be unaffected by a late-onset neurodegenerative disorder, being indicative of the molecular basis for the late-onset neurodegenerative disorder in the mammal to be diagnosed.

Another aspect of the present invention relates to a method for identifying a compound for mitigating ROS-induced damage associated with late-onset neurodegenerative disorder in a mammal. Decreased levels of AIF expression are associated with oxidative stress and cell death. Moreover, cells with decreased AIF protein levels are characterized by increased sensitivity to both exogenous and endogenous peroxides. These results are consistent with recent crystallization studies that reveal that the structure of AIF is highly similar to that of glutathione reductase, a potent antioxidant and scavenger of hydrogen peroxide, suggesting that AIF is also acting as a peroxide scavenger and anti-oxidant.

The method includes the steps of incubating an AIF mutant cell line, characterized as having decreased levels of AIF protein expression, or activity, with 1) an agent known to be responsible for ROS-induced damage (in a concentration effective to induce such damage), and 2) a compound to be tested for its ability to mitigate ROS-induced damage. Such a method is best practiced in a high-throughput format, the type of which is well-known to those skilled in the art. The AIF mutant cell line may be derived from any mammal of interest and, in preferred embodiments, is immortalized. Agents that cause ROS-induced damage can include, without limitation, hydrogen peroxide, glutamate, endogenous peroxides, or exogenous peroxides.

Another aspect of the present invention relates to a method for treating, or conferring resistance to, oxidative stress in cells of a mammal by providing an effective amount of wild-type AIF protein to the cell. As detailed in the Exemplification section that follows, such a method can also provide for increasing resistance of a cell to growth factor withdrawal-mediated cell death and preventing aberrant cell cycle re-entry associated with oxidative stress. Oxidative stress is associated with formation of cancerous tumors, in part due to cell cycle-re-entry.

In this embodiment, wild-type AIF protein is delivered into a cell using techniques known in the art. For example, a protein expression vector containing AIF coding sequence can be used to transfect cells. The expression vector can be, but not limited to, a retroviral vector. Preferably, the expression of AIF protein is targeted specifically to the mitochondria using the endogenous mitochondrial localization signal normally present in AIF protein. Alternatively, purified AIF protein, for example derived by recombinant DNA technique, may be delivered directly into a cell or to a specific tissue site, or administered systemically by modes of injection.

Another aspect of the present invention relates to a method for making a mutant cell line that is sensitive to oxidative stress, comprising manipulating the cell to effect a decrease in the level of AIF protein in the cell. As described above, such a cell line, characterized as having decreased AIF protein expression levels, can be used, for example, to identify compounds capable of mitigating ROS-induced damage. This method can be used to generate AIF mutant lines of different cell types.

AIF protein levels can be decreased in cells using techniques known in the art. For example, anti-sense RNA that is complimentary to Aif sequences can be delivered into cells such that, upon hybridization of anti-sense Aif RNA to sense Aif RNA, an effective reduction in AIF expression can be achieved.

Another aspect of the present invention also provides for a mutant cell line derived from a harlequin mouse. The mutant cell line can be made immortal, as for example with the use of SV40 large T antigen.

### EXEMPLIFICATION

### Example 1

Cerebellar Neuron Loss in Harlequin Mutant Mice

In contrast to the early postnatal death of granule and Purkinje cells caused by many cerebellar mouse mutations (reviewed in Sotelo and Wassef, Philos. Trans. R. Soc. Lond. B. Biol. Sci. 331: 307-313 (1991); Heintz and Zoghbi, Annu. Rev. Physiol. 62: 779-802 (2000)), later-onset cerebellar degeneration and truncal ataxia has been reported in mice hemizygous or homozygous for the harlequin (Hq) mutation (Bronson et al., Mouse Genome 87: 110 (1990)). To determine the onset of ataxia in Hq mutant mice, rotarod tests were performed on mutant and control mice. A significant reduction in the latency to fall was observed at 16 and 22 weeks in homozygous females and hemizygous males, respectively, when compared to age-matched controls. Younger mice did not exhibit overt locomotor abnormalities, nor did they demonstrate deficits in motor coordination in the rotarod assay. In addition, motor coordination defects were not observed in heterozygous females, even when aged to 24 months; nor were rotarod test values different between 43 week Hq/+ and +/+ females (254.6±16.8, n=17 vs. 214.1±19.3, n=10, respectively).

To determine if the onset of ataxia correlated with the loss of cerebellar neurons, cerebella from pre- and postataxic mutant mice were examined histologically. Prior to three months of age, cerebella of hemizygous or homozygous mutant mice were indistinguishable from those of littermate controls. However, at four months, many pyknotic granule cell nuclei were observed, and by seven months, mutant cerebella were approximately 33% the size of age-matched littermate controls. Granule cells were preferentially lost in the caudal (from lobules 6 through 10) vermis and hemispheres, although some granule cells in the rostral portion of the cerebellum also degenerated. Granule cells continued to die and by 12 months of age, the majority of granule cells in the caudal half of the cerebellum were lost.

To further investigate the nature of neuron loss in Hq mutant mice, TUNEL staining, which detects nicked DNA characteristic of apoptotic cells, and electron microscopic analysis were performed. TUNEL-positive granule cells were observed in four-month Hq mutant mice, but not in littermate controls. In addition, electron microscopic analysis demonstrated that dying granule cells had hallmarks of apoptosis, including nuclear condensation and blebbing.

Purkinje cells in Hq mutant mice also degenerate. However, unlike the early-onset cerebellar degeneration mutants reported to date in which granule cell death appears secondary to the loss of Purkinje cells (reviewed in Sotelo and Wassef, Philos. Trans. R. Soc. Lond. B. Biol. Sci. 331: 307-313 (1991); Heintz and Zoghbi, Annu. Rev. Physiol. 62: 779-802 (2000)), death of granule cells in Hq mutant mice occurs prior to Purkinje cell loss. At four months of age when many pyknotic granule cell nuclei are observed, almost all Purkinje cells are present. However, by seven months, many Purkinje cells have died and as with the granule cell death, this cell loss is higher in the caudal lobules of the cerebellum. Electron micrographs of dying Purkinje cells revealed swollen mitochondria and fragmented cell membranes, suggesting that unlike granule cells, these cells die via necrosis.

The Hq Mutation Disrupts the Apoptosis Inducing Factor Gene

To more finely localize the Hg mutation, 1152 offspring from an intersubspecific backcross was analyzed. This analysis defined the Hq critical region to 0.61±0.11 cM (7 recombinants/1152 meioses) between DXMit48 and DXMit82. YACs containing these markers were identified from the Whitehead/MIT Center for Genome Research set. A 610 Kb YAC (Y161_B^{—}12) containing both flanking markers, and thus spanning the interval, was identified.

The human genomic sequence homologous to this region of the mouse X chromosome contained several potential Hq candidate genes, whose mouse orthologs are: Elf4, an ETS-domain transcription factor (NM_{—}019680), apoptosis inducing factor (Aif; GenBank accession number, BC003292), Rab33a, a member of the RAS family of small GTPases (NM_{—}011228), Slc25a14, a brain mitochondrial carrier protein (NM_{—}011398), as well as the mouse ortholog of a putative zinc finger protein (AL133204) and a putative RNA binding protein (AL050405). PCR analysis confirmed that these genes were present on the YAC161_B_{—}12, and analysis of critical recombinants for single nucleotide polymorphisms present in the Slc25a14 3′UTR and intron 16 of the Aif gene, demonstrated that these genes co-segregate with the Hq mutation. However, neither expression differences nor cDNA polymorphisms between Hq mutant and wild type cerebellar RNA were detected in transcripts from the Elf4, Rab33a, Slc25a14, the zinc finger or RNA binding protein-encoding genes by northern analysis and sequencing of RT-PCR products, respectively.

In contrast, Aif transcripts were severely reduced in the two-month (pre-ataxic) Hq/Hq mutant cerebellum. Similarly, AIF protein levels were greatly reduced in the one month Hq/Hq cerebellum, the remainder of the brain, and the liver when compared to levels in these tissues in Hq/+ and +/+ females. However, Aif transcript and protein levels in tissues of Hq/+ mice were only slightly decreased from +/+ levels, consistent with the lack of cell death in heterozygous females.

To further test Aif as a candidate gene for the Hq locus, its cerebellar expression was examined. AIF is expressed in both granule and Purkinje cells, consistent with the pattern of neuron loss in mutant mice. However, RT-PCR products from Hq mutant brain RNA using Aif primers revealed no sequence differences from wild type message.

To look for obvious rearrangements of the Aif gene in the Hq genome that might lead to decreased expression, introns 2 through 16 were amplified by PCR from both mutant and wild type genomic DNA. No size differences in these amplified products were observed between Hq mutants and wild type controls. However, multiple restriction fragment length polymorphisms between Hq/Y and +/Y genomic DNA were detected by Southern blot analysis with a probe from intron 1. Notably, in EcoRI-digested genomic DNA, the mutant band was larger than that of the control (16 kb vs 7 kb, respectively). This suggested that the Hq genome had a 9 kb insertion in intron 1 of the Aif gene and that this insertion did not contain an EcoRI site. Murine ecotropic leukemia proviruses, which are endogenous retroviruses normally present at 1-2 copies/mouse genome, are approximately 8.8 kb in size and do not contain EcoRI restriction sites within their genome (Coffin et al., Ann. N Y Acad. Sci. 567: 39-49 (1989)). Therefore, it was investigated whether an ecotropic viral integration had occurred in the Aif gene of Hg mutant mice. Southern blots were rehybridized with a probe specific for the ecotropic proviral envelope gene, and bands that were identical in size to those detected with the intron-specific probe were observed in Hq, but not wild type, genomic DNA digested with several different enzymes.

To confirm an ecotropic proviral insertion, mutant and wild type genomic DNA was subjected to PCR analysis using pairs of primers designed to produce overlapping PCR products spanning the 7.7 kb intron 1. One primer pair (intron 1forward/1reverse), at bp 3262 and 3772 of intron 1, amplified products from control DNAs, but failed to amplify mutant DNA. Conversely, PCR reactions using this reverse intron primer and an ecotropic LTR-specific primer positioned 420 bp upstream of the 3′ end of the provirus, amplified a product from mutant, but not wild type, DNA. Sequencing confirmed this product contained LTR sequence juxtaposed to mouse genomic sequence beginning at bp 3432 of intron 1. To determine whether this provirus was unique to Hq mutant mice, PCR reactions with both primer pairs were repeated on genomic DNA from 25 inbred strains (see Methods). In all cases, the intron fragment was amplified and the LTR/intron reverse primer pair failed to generate products. Combined, these results demonstrate that the Hq genome contains an ecotropic proviral insertion in intron 1 of the Aif gene. Like many other proviral insertions, this insertion leads to a decrease in gene expression with approximately an 80% reduction in Aif mRNA and protein levels in the Hq mutant mice compared to wild type control levels.

Progressive Retinal Degeneration in the Harlequin Mouse Mutant

AIF is expressed in many tissues (Daugas et al., FEBS Letters 476: 118-123 (2000)), and immunofluorescence studies on CNS structures also demonstrated widespread AIF expression. In addition to cerebellar neurons, AIF is expressed in the hippocampus, dentate gyrus, olfactory bulb, cerebral cortex and various brainstem nuclei. However, in mice aged up to 26 months, neuron loss was not generally observed in non-cerebellar brain structures, although occasional mice had a thinning of the ventral surface of the dentate gyrus. Degeneration of the granule cells in dentate gyrus in these mutant mice was noted solely in the ventral surface and never proceeded around the hilus.

Expression of AIF was also noted in all cell layers of the wild type retina. When eyes were examined in aging Hq mutant mice, progressive retinal degeneration was observed. Differences in retinal morphology were not observed between Hq mutants and wild type control littermates prior to three months of age. At three months of age, Hq/Hq and Hq/Y mice exhibit a loss of some ganglion and amacrine cells in the periphery of the retina, while all other cell layers remain intact. However, in four-month old Hq/Hq and Hq/Y mice, cell loss in the ganglion cell and the inner and outer nuclear layers was observed, particularly in the peripheral retina. Cell loss continues as the mice age, and by 11 months, cell loss is very apparent in all regions of the retina, including the ganglion cell layer, which contains the nuclei of both ganglion and displaced amacrine cells; the inner nuclear layer, which contains the horizontal, bipolar and amacrine nuclei; and the outer nuclear layer where the cell bodies of rods and cones reside. In addition, the inner and outer plexiform layer (IPL and OPL, respectively), containing the axons and dendrites of these neurons, have thinned out considerably. Retinal neuron loss did not occur in Hq/+ mice, even those aged to 24 months.

Retinal neuronal loss was further defined by marker studies on mutant and wild type retinas. Loss of horizontal cells, large neurons found at the outer aspect of the inner nuclear layer, was revealed by immunohistochemistry with antibodies to calbindin-D28. Calbindin-expressing amacrine and ganglion cells are also clearly lost in aging mutant mice. To further differentiate amacrine and ganglion cells, sections were stained with antibodies to syntaxin, a specific marker of amacrine cells. This analysis demonstrated that amacrine cells within both the ganglion cell layer and the inner aspects of the inner nuclear layer begin to degenerate in the Hq mutant mice at four months and continue to die in the retinas of aging mutant mice. Immunohistochemistry with antibodies to 165 KDa neurofilament, specifically expressed in the ganglion cells in the retina, showed that this cell type is also progressively lost from the ganglion cell layer beginning at four months. In contrast, PKC-immunoreactive bipolar cells did not appear to vary between Hq mutant mice and wild type control littermates, suggesting a lack of death in this retinal cell type.

Ganglion cell loss in Hq mutant mice was further confirmed by the analysis of optic nerves from mutant and wild type mice. Ultrathin sections of these nerves revealed an approximate 50% reduction in size with many fewer axons than wild type control littermates. Electron microscopy revealed normal axonal morphology in wild type optic nerves. However, most of the remaining axons observed in the mutant mice were extremely swollen compared to wild type mice. The myelin sheaths in the swollen axons, however, were intact and the intracellular disorganization typical of dying axons (Shibuya et al., J. Vet. Med. Sci. 55: 905-912 (1993)) was not observed in axons of Hq mutant optic nerves.

Electroretinography (ERG) performed on Hq mutant mice and littermate controls confirmed the histological findings. Hq mice at five weeks of age show normal rod and cone responses. However, by four months of age, both the rod and cone ERG responses in Hq mutant mice diminish, although the shapes of the curves are normal. By 11 months, both the rod and cone responses are completely abolished in Hq mutant mice, while control littermates show normal ERG responses. Therefore, both clinically and histologically, Hq mutant mice exhibit later-onset retinal degeneration.

Loss of AIF Function Leads to Oxidative Stress

AIF is a mitochondrial oxidoreductase that translocates to the nucleus leading to nuclear condensation and apoptosis (Susin et al., Nature 397: 441-446 (1999)). In addition to its role in inducing apoptosis, AIF has potent redox function in vitro, which is separable from its apoptogenic activity (Miramar et al., J. Biol. Chem. 276: 16391-16398 (2001)). Many oxidoreductases have been shown to play important roles in maintaining intracellular free radical homeostasis (reviewed in Droge, W., Physiol. Rev. 82: 47-95 (2002)). Under conditions favoring excess free radical production, lipid peroxidation and oxidative damage of membranes and DNA may ensue, leading to events associated with later-onset neurodegenerative disorders.

To examine if a reduction in AIF leads to conditions of oxidative stress, antioxidant enzyme levels, lipid peroxidation and DNA oxidative damage in Hq and wild type mice were examined. Due to the similarity of the oxidoreductase moiety of AIF to bacterial hydrogen peroxide scavengers, it was hypothesized that changes in catalase and glutathione levels would occur in Hq mutant mice. Catalase is the major scavenger of hydrogen peroxide in cellular systems (reviewed in Deisseroth et al., Physiol. Rev. 50: 319-375 (1970)), while glutathione is an essential electron donor for the reduction of hydroperoxides (Baillie et al., Acc. Chem. Res. 24: 264-270 (1991)). In agreement with this, catalase activity is increased in the cerebella of Hq mutant mice at both 1 and 3 months of age compared to wild type levels. However, no differences were noted in the remainder of the brain at either one or three months (p>0.05). As with catalase, total glutathione levels were increased in the cerebella of Hg mutant mice at both one and three months of age compared to wild type levels. No differences were observed in total glutathione levels in the remainder of the brain at either one or three months (p>0.05). Western blot analysis of cerebellar extracts of one and three month old Hg mutant and wild type mice showed increases in catalase expression consistent with the increased catalase activity, while no differences in either SOD1 or SOD2 levels were observed at either age.

Increases in lipid hydroperoxides have been linked to oxidative stress in numerous human neurodegenerative disorders (reviewed in Sayre et al., Curr. Med. Chem. 8: 721-738 (2001)). In the Hq mutant mice, lipid hydroperoxides were increased in both brain and cerebellum at one and three months compared to wild type mice.

A small percentage of Hq mutant mice (5-10%) do not survive past two months of age. Upon necropsy of these mice, it appeared that several had enlarged hearts. To confirm and quantitate this finding, heart and body weight measurements were taken in wild type and mutant mice. The incidence of increased heart size was evident by a significant increase in the ratio of heart to body weight in Hq mutant mice. Lipid hydroperoxides were also increased in the hearts of Hq mutant mice at both one and three months compared to those of wild type mice. These findings are consistent with observations from animals undergoing oxidative stress, as well as those with certain mitochondrial disorders (Li et al., Nat. Genet. 11: 376-381 (1995); Esposito et al., Proc. Natl. Acad. Sci. USA 96: 4820-4825 (1999)).

Immunofluorescence using an antibody to 8hydroxydeoxyguanosine (8-OHdG), a major component of oxidatively damaged DNA, revealed positive neurons in both the cerebella and retinas of Hq mutant mice, but not of wild type littermates. Reactive cells were observed in the inner granule layer of the cerebellum, and the inner nuclear and ganglion cell layers of the retina, corresponding to areas of widespread cellular loss in the Hq mutant mouse. The majority of the DNA staining positive for 8-OHdG is non-nuclear, consistent with oxidative damage in the mitochondria. 8-OHdG-positive Purkinje cells and photoreceptors of the retinal outer nuclear layer were not observed.

Oxidative Stress Induces Cell Cycle Re-Entry and Apoptosis

Although oxidative stress has been observed in neurons from patients with several human neurodegenerative disorders, the means by which such stress leads to cell death remain poorly understood. Similarly, cell cycle abnormalities have been reported in neurons in brain regions undergoing degeneration, yet the mechanisms causing cell cycle re-entry of terminally-differentiated neurons are unknown. Because oxidative stress has been associated with the formation of cancerous tumors, in part due to cell cycle re-entry or abnormal cell cycle checkpoint function (reviewed in Shackelford et al., Free Radical Biology & Medicine 28: 1387-1404 (2000)), whether cell cycle control is disrupted in Hq mutant neurons was investigated.

Hq/Hq mice and Hq/+ littermate controls were injected with bromodeoxyuridine (BrdU) at 3, 4, 5, 7, 9, and 12 months to mark cells which have undergone DNA synthesis. Mice were sacrificed 20 hours post-injection, and immunohistochemical analysis of 20 adjacent midline sagittal sections was performed. Virtually no labeled granule cells were found in Hq/+ littermate controls; however, many BrdU-positive granule cells were seen in Hq/Hq cerebella. Aberrant cell cycle re-entry was further confirmed by immunofluorescence with antibodies against proliferating cell nuclear antigen (PCNA) or CDC47, both expressed during S phase through the late G2 phase of the cell cycle, but not in quiescent cells (Kurki et al., Exp. Cell Res. 166: 209219 (1986); Kimura et al., Genes Cells 1: 977-993 (1996)). As observed in the BrdU studies, many granule cells in the cerebella of older Hq/Hq females, but not cells from aged-matched heterozygotes, were reactive with these antibodies, further confirming cell cycle misregulation in mutant cells. Double-labeled cells were not detected in co-immunofluorescence experiments using antibodies to PCNA and the glial marker, glial fibrillary acidic protein (GFAP), confirming that cycling cells were not glia.

The number of BrdU-positive granule cells steadily increased through seven months of age then began declining, likely due to the extensive loss of many granule cells by nine and 12 months of age. In addition, more BrdU-positive cells were located in the caudal region of the cerebellum, consistent with findings of increased cell death in the caudal region of the Hq mutant cerebellum. However, BrdU-positive Purkinje cells were not observed in these mice, nor were BrdU-positive neurons present in littermate controls. In summary, these data demonstrate that Hq mutant granule cells, but not Purkinje cells, re-enter the cell cycle during the time when degeneration is occurring.

In the retina, BrdU- and PCNA-positive ganglion, amacrine and horizontal cells were observed in the Hq mutant mice, but not in wild type control littermates. Similar to the cerebellar data, abnormally cycling retinal neurons were first detected at approximately four months of age and numbers had increased at seven months, but then began declining as the total population of ganglion, amacrine and horizontal cells decreased.

Many of the cells that incorporated BrdU had pyknotic, blebbing, or fragmented nuclei typical of cells undergoing apoptosis. In agreement, when cerebellar and retinal sections from mutants were double labeled with antibodies against the cell cycle marker CDC47 and caspase 3, an initiator of neuronal apoptosis, all cells positive for caspase 3 were also positive for CDC47. Furthermore, a subpopulation of CDC47-positive cells was not positive for caspase 3, suggesting cell cycle re-entry precedes neuronal apoptosis in both the cerebellum and retina, consistent with previous reports from both mouse and human studies (Herrup and Busser, Development 121: 2385-2395 (1995); Migheli et al., Am. J. Pathol. 155: 365-373 (1999); Frade, J. M., J. Cell Sci. 113: 1139-1148 (2000); Raina et al., Prog. Cell Cycle Res. 4: 235-242 (2000)). In the retina, as in the cerebellum, all cells positive for CDC47 were also positive for caspase 3. However, no CDC47 or caspase 3-positive Purkinje, or photoreceptor cells, were observed.

To examine a potential correlation between oxidative stress and apoptosis, sections from mutant cerebellum and retina were double-labeled with antibodies against 8-OHdG and caspase 3. Double-positive cells were observed in ganglion, horizontal and amacrine cells of the Hq mutant retina. Furthermore, granule cells reactive with both antibodies were noted in the Hq mutant cerebellum. All caspase 3-positive cells were positive for 8-OHdG in both cerebellum and retina. No cells positive for either 8-OHdG or caspase 3 were noted in the wild type retina or cerebellum. As with the staining for CDC47 and caspase 3, a subpopulation of cells in both the retina and the cerebellum of Hq mutant mice were positive for 8-OHdG, but not caspase 3, suggesting that oxidative stress precedes apoptosis in these cells.

To determine if there was a direct correlation between oxidative stress and cell cycle re-entry, sections from mutant cerebellum and retina were double-labeled with antibodies against 8-OHdG and CDC47. All CDC47-positive cells in both the retina and cerebellum were also reactive with 8-OHdG antibodies, demonstrating a tight correlation between oxidative stress and cell cycle re-entry. However, 8-OHdG-positive neurons that were not reactive for CDC47 were also observed suggesting a temporal relationship between oxidative stress and cell cycle re-entry, with oxidative damage preceding cell cycle re-entry.

Methods

Genetic Mapping.

Hq arose on a CF1 outbred stock and was transferred to a B6CBACa-A^{w-J}/A (B6CBA) background. As previously reported, hemizygous males (Hq/Y) and homozygous females (Hq/Hq) are nearly bald, while Hq/+ females have patchy hair loss (Barber, B. R., Mouse News Letter 45: 35 (1971)), allowing identification of mutant mice by weaning age.

High-resolution genetic mapping of the Hq mutation was performed by typing N2 progeny of a (B6CBACa-A^{w-J}/A-Hq/Hq X CAST/Ei) X B6CBACa-A^{w-J}/A-Hq/Y backcross with SSLP markers (MIT/Whitehead Center for Genomics) on the X Chromosome.

Rotarod Tests and ERG Measurements.

Mice were placed on a rotarod (Ugo Basile, Comerio, Italy), accelerating linearly from 4-40 rpm over a 5-minute period. The latency to fall was averaged from the results of four trials. A test was considered completed when the mouse fell from the rotarod, 300 seconds expired, or the mouse used gripping behavior to remain on the rod. Statistical significance was determined by two-way ANOVA (genotype×age). Tukey HSD test was used for post hoc analysis of significant main effects. Full field ERG was performed as previously described (Hawes et al., Invest Ophthalmol. Vis. Sci. 41: 3149-3157 (2000)).

Immunohistochemistry and Electron Microscopy.

Mice were anesthetized with tribromoethanol and transcardially perfused with 4% paraformaldehyde in phosphate-buffered saline. TUNEL assays were performed using the in situ cell death detection kit (Roche Diagnostics, Indianapolis, Ind.) as per manufacturer's protocol. Immunohistochemistry was performed as previously described using antibodies to Calbindin-D28 (Swant, 1:1500) and colorimetric detection with DAB (Ackerman et al., Nature 386: 838-842 (1997)). For immunofluorescence studies, processed paraffin sections were incubated at 4° C. overnight with the mouse monoclonal antibodies to: BrdU (1:50; Dako Corporation, Carpenteria, Calif.), PCNA (1:50; Santa Cruz Biotechnology, Santa Cruz, Calif.), CDC47 (1:50; NeoMarkers, Fremont, Calif.), 8-OHdG (1:1000; QED Bioscience, San Diego, Calif.) neurofilament 165 kD (1:50; DSHB, Iowa City, Iowa), and rabbit polyclonals to: caspase 3 (1:50; NeoMarkers, Fremont, Calif.), PKC (Cambio, Cambridge, UK; 1:2000) and GFAP (1:50; Dako Corporation, Carpinteria, Calif.). To distinguish between mouse monoclonal antibodies, antigen subtype-specific goat secondary antibodies were used against either mouse IgG1 or IgG2a (Southern Biotechnology, Birmingham, Ala.). The mouse monoclonal antibody to syntaxin (1:500, Sigma, St. Louis, Mo.) was used on cryosections of Z-fixed (Anatech Ltd, Battle Creek Mich.) retina. Expression was visualized with Cy-3 or FITC labeled donkey or goat secondary antibodies to mouse or rabbit immunoglobulins (1:200, Jackson Immunoresearch, West Grove, PA). Optic nerves were stained with p-phenylenediamine, embedded in resin and sectioned at 1 μm. Electron microscopic studies were performed on osmium tetroxide/lead citrate-stained ultrathin sections as previously reported (Lynn et al., J. Auton. Nerv. Syst. 62: 174-182 (1997)).

PCR Analysis.

Intron 1 of the Aif gene was isolated from a BAC clone (RP23-305N1) using specific primers to exons 1 and 2 with the Expand Long Template PCR System (Roche Diagnostics, Indianapolis, Ind.), as per manufacturer's protocol, and sequenced.

To determine the ectopic proviral insertion site, PCR analysis on genomic DNA from Hq mutant mice, B6CBA littermate controls, and CF1 mice was performed using forward and reverse primers from intron 1 of Aif (5′-AGTGTCCAGTCAAAGTACCGGG-3′ and 5′-CTATGCCCTTCTCCATGTAGTT-3′, respectively) and a viral primer from the U3 region of the LTR from murine C-type ecotropic virus (Genbank accession U63133; 5′-CCAGAAACTGTCTCAAGGTTCC-3′). PCR was done according to standard procedures with 40 cycles of 30s 94° C., 45s 60° C., and 45s 72° C., and products were electrophoresed through a 2% agarose gel. Other strains genotyped for this insertion were: A/J, AKR/J, AU/SsJ, BALB/cByJ, BUB/BnJ, CAST/Ei, CBA/J, C3HeB/FeJ, C57BL/6J, C57BLKs/J, CZECHII/Ei, DBA/2J, DA/HuSn, F/St, FVB/NJ, NOD/LtJ, NZB/BlNJ, NZW/LacJ, RIIIS/J, SIM/Ut, SJL/J, SWR/J, and WSB/Ei.

Southern Blot Analysis.

Southern blots were hybridized to ³²P-labeled probes corresponding to a 1200 bp fragment amplified using a reverse primer from exon 2 (5′-CCTGATGAACCAGAGCTAGC-3′) and a forward primer 1200 bases upstream in intron 1 (5′-GTATGTCATGGCTGTGTGCT-3′); or to the envelope protein (pEcB4) of the AKV class of murine ecotropic virus (ENV probe) as described (Taylor and Rowe, Genomics 5: 221-232 (1989)).

Western Blotting.

Brain extracts were prepared as previously described (Sheldon et al., Nature 389: 730-733 (1997)). Blots were incubated with goat anti-AIF antibody (1:1000, Santa Cruz Biotechnology), rabbit anti-catalase (1:2000, Rockland, Gilbertsville Pa.), sheep anti-SOD1 (1:1000, Upstate Biotechnology, Lake Placid, N.Y.), goat anti-SOD2 (1:1000, Upstate Biotechnology), or rabbit anti-human neuron specific enolase (1:1,000, Scytek Laboratories, Logan, Utah) prior to incubation with HRP-conjugated goat, rabbit or sheep secondary antibodies (Santa Cruz or Upstate Biotechnology; 1:1,000 dilution). Blots were developed using the ECL kit (Amersham).

Lipid Peroxidation.

Lipid hydroperoxides were extracted from equivalent amounts of tissue using the degassed acid-methanol/chloroform extraction protocol as per the manufacturer's procedure (Cayman Chemical Company, Ann Arbor, Mich.). All samples and standards were assayed in triplicate at 500 nm in a spectrophotometer as per manufacturer's protocol. Statistical significance was determined by t-test.

Catalase and Glutathione Assays.

Proteins were extracted in western lysis buffer and quantitated by Bradford assay (Sigma, St Louis, Mo.). Catalase activity was determined in triplicate at 560 nm using the Amplex Red Catalase Assay Kit as per the manufacturer's protocol (Molecular Probes, Eugene, Oreg.). Statistical significance was determined by t-test.

Equivalent extract volumes to those used in the catalase assay were deproteinated using metaphosphoric acid (Aldrich Chemical Company, Milwaukee, Wis.) prior to performing the glutathione assay (Cayman Chemical Company, Ann Arbor, Mich.). All samples and standards were run in triplicate at 405 nm as per manufacturer's protocol. Statistical significance was determined by t-test.

### Example 2

Decrease in AIF Levels in Neuronal Granule Cells Confers Sensitivity to Both Exogenous and Endogenous Peroxides

To determine if decrease in AIF levels leads to increased sensitivity to exogenous peroxides, primary neuronal granule cell cultures from Hq mutant mice were treated with increasing doses of hydrogen peroxide (0, 5, 20, 50 mM) for 5 hours and then stained with propidium iodide (PI) to identify apoptotic cells. Significantly more cell death was observed in cultures of Hq mutant cells treated with 20 and 50 mM of hydrogen peroxide than in cultures derived from wild-type control mice. No differences between the viability of wild type and Hq granule cells were observed in normal culture conditions.

To determine if decrease in AIF levels leads to increased sensitivity to endogenous peroxides, primary neuronal granule cell cultures from Hq mutant mice were treated with 100 mM and 2000 mM glutamate. Low doses of glutamate cause neuronal excitotoxicity via NMDA receptors, while higher doses of glutamate trigger intracellular oxidative stress through the release of endogenous peroxides. Cell viability was similar between mutant and wild type granule cells treated with low concentrations of glutamate, while higher doses produced significant increases in Hq mutant granule cell death.

These results demonstrate that down regulation of AIF confers sensitivity to both exogenous and endogenous peroxides. The results are consistent with recent crystallization studies that reveal that the structure of AIF is highly similar to that of glutathione reductase, a potent antioxidant and scavenger of hydrogen peroxide, suggesting AIF is also acting as a peroxide scavenger.

Neuronal Cell Peroxide Sensitivity to Decreased AIF Levels is Cell-Specific

To determine if increased peroxide sensitivity due to loss of wild-type levels of AIF is cell-specific, primary cultures of mutant and wildtype embryonic cortical cells were treated under the identical conditions used for granule cell cultures. Cell viability at all peroxide concentrations was similar to that observed in untreated controls, suggesting that cortical neurons, in general, are more resistant to hydrogen peroxide. When wild type and Hq mutant cortical neurons were treated with hydrogen peroxide for 24 hours, cell viability was comparable to values reported in the literature for similar culture conditions. In contrast to granule cells, no difference in viability was noted between mutant and wild type corticol cells at any peroxide concentration, consistent with the lack of neuronal death in the cortex of aged Hq mutant animals.

Decrease in AIF Levels in Neuronal Granule Cells Confers Resistance to Growth Factor Withdrawal-Mediated Apoptosis

To determine whether decreased AIF levels in granule cells confers resistance to growth factor withdrawal mediated cell death, primary granule cell cultures were serum starved. Following 5 hours of serum starvation, significantly less cell death was observed in mutant granule cell cultures compared to wild type cultures, confirming the role of AIF in mediating growth factor withdrawal-mediated apoptosis in neurons. In contrast, etoposide (50 mM) or staurosporine (0.5 mM) treatment for 24 hours resulted in no significant difference in cell death between Hq mutant and wild type granule cells.

These results confirm the role of AIF in mediating growth factor withdrawal-mediated apoptosis (non-peroxide mediated cell death) in neurons.

AIF Overexpression in Granule Cells Confers Peroxide Resistance

To confirm that the peroxide sensitivity of Hq mutant granule cells is indeed due to downregulation of AIF, granule cells were infected with murine stem cell retrovirus expressing the Aif coding sequence upstream of an IRES/green fluorescent protein (GFP), or the retrovirus without Aif sequences for three days prior to peroxide exposure and subsequent propidium iodine staining. The proportion of dead to live cells was determined from both infected (GFP-positive; approximately 50% of each culture) and uninfected (GFP-negative) cells. First, no differences in cell viability were observed between infected (GFP-expressing) mutant neurons and uninfected (GFP-negative) wild type cells at any concentration of peroxide, while non-infected Hq neurons retained their peroxide sensitivity when treated with either 20 or 50 um concentrations. Second, overexpression of AIF in wild type neurons resulted in increased peroxide-resistance. No difference in viability was observed between uninfected wild type or Hq mutant granule cells and cells infected with control retrovirus expressing GFP, but not AIF. These data suggest that both wild type and Hq mutant granule cells were significantly more resistant to hydrogen peroxide, while cells not infected by the retrovirus were as sensitive as previously reported above.

The results of this disclosure demonstrate that in addition to its apoptogenic role, AIF has antioxidant activity, particularly in regards to peroxide scavenging.

1. A method for diagnosing the molecular basis for a late-onset neurodegenerative disorder in a mammal, the method comprising:
a) providing a sample from the mammal, the sample containing a nucleic acid sequence encoding the AIF protein;
b) determining the sequence of nucleic acids in the nucleic acid sequence of step a);
c) comparing the sequence of nucleic acids determined in step b) to a nucleic acid sequence encoding the wild-type AIF protein, a difference between the sequence of nucleic acids determined in step b), and the nucleic acid sequence encoding the wildtype AIF protein, representing a candidate mutation; and
d) further analyzing the candidate mutation for an association with a decrease in expression of functional AIF protein, such association confirming a molecular basis for the late-onset neurodegenerative disorder in the mammal.

2. The method of claim 1 wherein the mammal is a human.

3. The method of claim 1 wherein the late-onset neurodegenerative disorder is characterized by accumulation of ROS-damaged proteins.

4. A method for diagnosing the molecular basis for a late-onset neurodegenerative disorder in a mammal, the method comprising:
a) providing a biopsy sample comprising neuronal cells;
b) determining expression level of AIF protein in the cells of step a); and
c) comparing the level determined in step b) to an otherwise identical determination from the cells of an individual known to be unaffected by a late-onset neurodegenerative disorder, a substantial decrease in the level of expression of the AIF protein in the cells of the mammal to be diagnosed, as compared to the level from the cells of the mammal known to be unaffected by a late-onset neurodegenerative disorder, being indicative of the molecular basis for the late-onset neurodegenerative disorder in the mammal to be diagnosed.

5. The method of claim 4 wherein the biopsy is a liver biopsy.

6. The method of claim 4 wherein the biopsy is postmortem.

7. The method of claim 4 wherein the mammal is human.

8. The method of claim 4 wherein the level of expression is determined by assaying transcript levels.

9. The method of claim 4 wherein the level of expression is determined by assaying protein levels.

10. The method of claim 9 wherein the protein levels are assayed using a monoclonal antibody which binds specifically to the AIF protein.

11. A method for identifying a compound for mitigating ROS-induced damage associated with a late-onset neurodegenerative disorder in a mammal, the method comprising:
a) providing a mutant cell line having decreased AIF protein activity;
b) incubating the cells from step a) with:
i) a compound to be tested for its ability to mitigate ROS-induced damage associated with late-onset neurodegenerative disorder in a mammal; and
ii) an agent known to induce ROS damage at a concentration sufficient to induce ROS damage; and
c) identifying a compound which mitigates ROS-induced damage by comparing the results of step b) with an otherwise identical step in which the compound to be tested is omitted.

12. The method of claim 11 wherein the mammal is human.

13. The method of claim 11 wherein the mutant cell line is produced from the cells of a harlequin mouse.

14. The method of claim 13 wherein the mutant cell line is immortal.

15. A method for treating oxidative stress in a cell of a mammal, the method comprising introducing an effective concentration of a wild-type AIF protein to the cell.

16. The method of claim 15 wherein introducing an effective concentration of the wild-type AIF protein is achieved by introducing an expression vector encoding wild-type AIF into the cell.

17. The method of claim 15 wherein expression of the wildtype AIF protein is targeted to the mitochondria.

18. The method of claim 15 wherein the mammal is human.

19. A method for making a mutant cell line sensitive to oxidative stress, the method comprising:
a) providing a cell line expressing a wild-type level of AIF protein activity; and
b) decreasing the wild-type level of AIF protein activity of the cell line in step a).

20. A mutant cell line sensitive to oxidative stress, the mutant cell line derived from a harlequin mouse.

21. A cell line according to claim 23 that is immortalized.
